(19) United States
(12) Patent Application Publication
(10) Pub. No.: US 2019/0091362 A1
(43) Pub. Date: Mar. 28, 2019

(54) MALODOR ABATEMENT IN WASTE PRODUCTS

(71) Applicant: Rohm and Haas Company, Collegeville, PA (US)

(72) Inventors: Andrea C. Keenan, Pottstown, PA (US); Theodore Tysak, Ambler, PA (US); Melissa S. Leach, Birdsboro, PA (US); Michelle Gallagher, Cherry Hill, NJ (US)

(21) Appl. No.: 16/086,332

(22) PCT Filed: Mar. 16, 2017
(86) PCT No.: PCT/US2017/022625

§ 371 Date: Sep. 19, 2018

Related U.S. Application Data
(60) Provisional application No. 62/316,022, filed on Mar. 31, 2016.

(51) Int. Cl. A61L 9/014; B01J 39/20; B01J 39/07; B01J 39/05; B01J 20/12; B01D 53/02; B01D 53/58; A01K 1/015

(52) U.S. Cl. CPC A61L 9/014; B01J 39/20; B01J 39/07; B01J 39/05; B01J 20/12; B01D 2257/90; B01D 53/58; A01K 1/0152; A61L 2209/22; B01D 2253/206; B01D 2257/406; B01D 53/02

(57) ABSTRACT

A method for reducing odor in waste products by adding an acidic ion exchange resin to an absorbent which is in contact with said waste products.

### BACKGROUND

This invention relates generally to a method for reducing odor in waste products such as cat litter used in cat litter boxes.

Cat litter boxes often have a strong odor of cat urine. Traditionally, cat litter boxes have odor control agents that will abate the strong pungent malodor from cat urine, which is believed to be due largely to ammonia and amines. This is typically achieved by using clay, charcoal, baking soda, odorized crystals or other adsorbing materials. If kept in room with an intake vent, an air freshener may be added on the furnace filter to isolate the odor from the rest of the house. The prior art discloses various treatments of clay to improve its efficiency, e.g., in U.S. Pat. No. 5,143,023. However, the prior art teaches that clay and silica are preferable to synthetic materials.

The problem addressed by this invention is to find an improved method for reducing odor in cat litter.

### STATEMENT OF INVENTION

The present invention is directed to a method for reducing odor in waste products by adding an acidic ion exchange resin to an absorbent which is in contact with said waste products.

### DETAILED DESCRIPTION

All percentages are weight percentages (wt %), and all temperatures are in ° C., unless otherwise indicated. All operations were performed at room temperature (20-25° C.), unless otherwise specified. Weight percentages of ion exchange resin are based on dry resin. The term “waste products” refers to feces, urine, sweat and other malodorous products excreted by humans or animals. The term “absorbent” refers to cat litter, shoe inserts, disposable diapers, incontinence pads, sanitary napkins, panty liners, mattress covers, air gels, carpets and fabrics.

Cat litter is absorbent material, often in a granular form that is used to line a receptacle in which a domestic cat can urinate and defecate indoors. There are many different types of cat litters available, but essentially most of them fall into three distinct categories: clay-based, silica-based, and biodegradable. Clay-based litters are largely absorbent clay material, often with small amounts of limestone, crystallized silica, sodium tetraborate, or a combination thereof. Silica-based litters are largely crystallized silica. Biodegradable litters are made from various plant resources, including pine wood pellets, recycled newspaper, clumping sawdust, Brazilian cassava, corn, wheat, walnuts, barley, okara and dried orange peel.

The term “acrylic resin” refers to a polymer having at least 70 wt % polymerized units of acrylic monomers, preferably at least 80 wt %, preferably at least 90 wt %, preferably at least 95 wt %, preferably at least 98 wt %, preferably at least 99 wt %. Acrylic monomers include (meth)acrylic acids and their C₁-C₂₂ alkyl, hydroxyalkyl or polyethylene glycol esters; crotonic acid, itaconic acid, fumaric acid, maleic acid, maleic anhydride, (meth)acrylamides, (meth)acrylonitrile and alkyl or hydroxyalkyl esters of crotonic acid, itaconic acid, fumaric acid or maleic acid.

Preferably, the ion exchange resin is in the form of substantially spherical beads. The ion exchange resin used in the present invention can be a gel-type resin or a macroreticular resin. A macroreticular resin is a resin having a surface area from 25 m²/g to 200 m²/g and an average pore diameter from 50 Å to 500 Å; preferably a surface area from 30 m²/g to 80 m²/g and an average pore diameter from 100 Å to 300 Å. Suitable resins include, e.g., acrylic resins, styrenic resins, and combinations thereof. Resins contain polymerized units of a multiethylenically unsaturated monomer (crosslinker). Preferably, the level of crosslinker in the resin is from 0.5% to 16 wt %, preferably at least 1%, preferably at least 2%; preferably no more than 14%, preferably no more than 12 wt %. Gel resins preferably have a crosslinker level of 0.5% to 4%. Macroreticular resins preferably have a crosslinker level of 3.5% to 16%.

In a preferred embodiment, the resin is an acrylic resin, typically containing 88% to 99.5% monomer residues of (meth)acrylic acid and 0.5% to 12% residues of a cross-linker, preferably 88% to 96.5% monomer residues of (meth)acrylic acid and 3.5% to 12% residues of a cross-linker, preferably 96% to 99.5% monomer residues of (meth)acrylic acid and 0.5% to 4% residues of a cross-linker, preferably divinylbenzene (DVB). Preferably, the average particle size of the gel resin is from 30 μm to 2000 μm, preferably at least 50 μm, preferably at least 100 μm; preferably no greater than 800 μm, preferably no greater than 500 μm. In a preferred embodiment, the ion exchange resin comprises polymerized units of styrene and a crosslinker, e.g., divinyl aromatics; di-, tri- and tetra-(meth)acrylates or (meth)acrylamides; di-, tri- and tetra-allyl ethers and esters; polyallyl and polyvinyl ethers of glycols and polyols. Preferably, the crosslinker is diethylenically unsaturated, e.g., DVB. Preferably, the acid functionality of the ion exchange resin comprises sulfonic acid groups, carboxylic acid groups, phosphoric acid groups or a mixture thereof; preferably sulfonic or carboxylic acids. A typical acidic ion exchange resin has from 0.4 to 8 meq/ml acid functionality, on a dry basis, preferably at least 2 meq/ml, preferably at least 3 meq/ml; preferably no more than 6 meq/ml. Preferably, the acid functionality is in the form of sulfonic acid groups.

Preferably, a weak acid ion exchange resin is used in the present invention. Preferably, the resin is functionalized with carboxyl groups, phosphonic acid groups, phosphoric acid groups, phosphinic acid groups, or a combination thereof; preferably carboxyl groups. In a preferred embodiment of the invention, a strong acid resin having sulfonic acid groups is used.

Preferably, the acidic ion exchange resin is added to the absorbent in an amount from 0.1 to 50 wt % of the absorbent plus ion exchange resin, preferably at least 0.5 wt %, preferably at least 1.0 wt %, preferably at least 3.0 wt %; preferably no more than 25 wt %, preferably no more than 10 wt %, preferably no more than 7 wt %.

### Experimental Procedures

5M ammonium hydroxide solution (corresponds to 8.77% ammonia) and 28% ammonium hydroxide solutions were purchased from Sigma Aldrich to make the ammonia standards. The stock solution was diluted with MilliQ water to make 10 ppm to 10% wt/wt standards of ammonium hydroxide. The weight concentrations were then converted to volume/volume (v/v) concentrations using the ideal gas law. Approximately 15 mg of each calibration standard was placed into a 22 mL headspace vial and capped with a Teflon-lined septum and then analyzed by headspace sampling combined with gas chromatography with mass selective detection (HS-GC-MS). The instrumentation was an Agilent GC-MS model 6890/5973 equipped with a Tekmar 7000 headspace autosampler with Silcosteel treated loop and connectors to minimize absorption. The column used for the separation was a ChromPac PoraPlot Amine column (25 m×0.32 mm×10 μm). The headspace analysis of the standards was done in a full-evaporation mode to eliminate matrix effects that can occur in static headspace sampling. In this mode, a small sample size is used, and the headspace vial temperature is set sufficiently high enough to allow for full evaporation of the volatile of interest. For this analysis, the standard samples were heated to 150° C. for 10 minutes prior to sampling. This temperature should fully liberate the ammonia from the ammonium hydroxide solution into the headspace. A calibration plot for ammonia was constructed (peak area m/z=17 vs. v/v concentration of ammonia). The m/z=17 ion was used for enhanced sensitivity.

To prepare the samples, the kitty litter and/or active were weighed into 22 mL headspace vials. Replicate samples were always prepared. Ammonia was added to each headspace vial containing the samples, as well as empty vials (to be used as controls), by dispensing a known volume of ammonia gas from either the headspace above an 8.77% or 28% ammonium hydroxide solution using an appropriate VICI gas-tight syringe, following by quickly capping the vial with a Teflon-lined septum. The vials were left at room temperature for the designated length of time and then the headspace in each vial was heated to 30° C. for 0 or 10 minutes and analyzed for ammonia content by HS-GC-MS. The concentration of ammonia in the headspace above this solution at room temperature was determined using the linear-least-squares equation from the calibration plot for ammonia.

### Examples

Diluted ammonia gas (approximately 500 ppm and 3000 ppm v/v in air) was added to 22 mL headspace vials containing samples of kitty litter and IER (see Table 1). The headspace ammonia was then measured in each headspace vial after 4 hours at room temperature to measure the ammonia abatement (run in duplicate). The dual sets indicate that this data is very repeatable. In this set of studies, the cat litters were at 0.1 grams and the weakly acidic cationic ion exchange resin (methacrylic acid/DVB, macroreticular) was tested at 0.1 g and 0.005 g.

TABLE 1

| | Avg ppm v/v Ammonia, | |
|---|---|---|
| Sample | 2 runs | % Abatement |
| Control | 3042 | |
| Commercial Fresh Step | 136 | 95.5 |
| Commercial Scoop Away | 50 | 98.4 |
| Commercial Arm and | 26 | 99.2 |
| Hammer | | |
| Weakly acidic IER 0.1 g | 2 | 99.9 |
| Weakly acid IER 0.005 g | 2 | 99.9 |
| Styrene Acrylates Copolymer | 2620 | 13.9 |

FRESH STEP (FS) comprises >80% Bentonite clay, <6% crystallized silica and 0.1-1% sodium tetraborate; SCOOP AWAY (SA) comprises 70-90% clay, 10-25% limestone, <6% crystallized silica and 0.1-1% sodium tetraborate; ARM & HAMMER (AH) comprises corn, pine and cedar; and a polymer that is a styrene-acrylic polymer with no acidic or basic functionality.

In all cases, 0.1 g of Kitty litter was weighed into the vials. Table 2 below shows spiking initially, a second time one day later, and re-capping the vial and waiting 3 days before retesting to determine if any ammonia is still present. Addition of the weakly acidic cationic IER at two different levels is able to abate ammonia after 3 days. This example served to show a similar scenario of leaving cats at home alone for a long weekend. There is significant differentiation for the ion exchange resin at shorter abatement times.

TABLE 2

| | | | | Weakly | Weakly |
|---|---|---|---|---|---|
| | | | | Acidic IER | Acidic IER |
| Sample* | FS | SA | AH | 0.1 g | 0.005 g |
| Initial 3000 ppm NH3, | 136 | 50 | 26 | 2 | 2 |
| 15 min lapse | | | | | |
| Re-spike 3000 ppm | 348 | 224 | 93 | 6 | 20 |
| NH3 | | | | | |
| Re-capped, rechecked | 75 | 29 | 4 | 0 | 0 |
| 3 days later | | | | | |

*All data reported is the average ppm v/v Ammonia (minimum 2 runs)

In Table 3, continuation of the odor abatement of the ion exchange resins was conducted with re-spiking of ammonia into the same vial (3000 ppm ammonia per spike, ultimately building up to 18000 ppm in spike 6). The re-spiking up to 6 times indicated that even with high ammonia levels (up to 18000 ppm ammonia), that the ion exchange resin still abates the ammonia odor.

TABLE 3

| | FS | Weakly Acidic IER 0.005 g | Weakly Acidic IER 0.001 g |
|---|---|---|---|
| Spike 1 | 80 | 3 | 2 |
| Spike 2 | 324 | 13 | 42 |
| Spike 3 | 603 | 29 | 82 |
| Spike 4 | 854 | 42 | 112 |
| Spike 5 | 1569 | 53 | 145 |
| Spike 6 | 1647 | 116 | 177 |

*All data reported is the average ppm v/v Ammonia (minimum 2 runs)

In Table 4, each vial in the series was spiked at the stated ammonia dosage, then examined for ammonia abatement. Addition of the ion exchange resin to the kitty litter does improve the capture of malodors. This effect would be significant with multiple cats or if customers leave cats alone for extended periods of time.

TABLE 4

| | | | | Weakly | FS and Weakly | | FS and |
|---|---|---|---|---|---|---|---|
| | Ammonia | | | Acidic | Acidic | Weakly | Weakly |
| | *Dosage | | | IER | IER | acidic IER | acidic IER |
| | ppm | Control | FS | 0.005 g | 0.005 g | 0.0025 g | 0.0025 g |
| Series 1 | 3000 | 2858 | 90 | 1 | 3 | 11 | 3 |
| Series 2 | 6000 | 5225 | 637 | 22 | 2 | 10 | 22 |
| Series 3 | 9000 | 7646 | 1131 | 16 | 4 | 68 | 11 |
| Series 4 | 12000 | 9608 | 1039 | 20 | 10 | 1042 | 43 |
| Series 5 | 15000 | 11734 | 1680 | 49 | 17 | 1413 | 111 |
| Series 6 | 18000 | 13095 | 2460 | 15 | 21 | 2141 | 186 |

In Table 5, we reviewed several different types of ion exchange resins for their ability to abate ammonia. The samples were spiked with 3000 ppm ammonia and allowed a 15 min abatement period prior to being analyzed. The data indicates the strong to weak acid cationic ion exchange resins have excellent odor abatement compared to strong base anionic ion exchange resins or nonionic ion exchange resins. Ammonia can be removed from air and liquids with a strong acid cation exchange resin or a weak acid cation exchange resin. When ammonia is present as the free base, a weak acid cation exchanger resin is preferred due to its higher capacity and higher regeneration efficiency. But a weak acid cation resin will only work when the ammonia is present as the free base; if it is present as a salt, a strong acid cation resin is needed to split the salt.

TABLE 5

| | | Ammonia, | | |
|---|---|---|---|---|
| | | ppm, v/v | | |
| | | (AVG), 2 | % | |
| | | runs | Abatement | Comments |
| Amt | Control | 3440 | 0 | Control |
| 0.1 | Fresh Step | 138 | 96.0 | Control |
| 0.0025 | Strong acid | 0 | 100.0 | Great |
| | cationic IER | | | |
| 0.0025 | Strong base | 3276 | 4.8 | Poor |
| | anionic IER | | | |
| 0.0025 | Strong acid | 0 | 100.0 | Great |
| | cationic IER | | | |
| 0.0025 | Divinylbenzene | 3358 | 2.4 | Poor |
| | hydrophobic | | | |
| | polyaromatic | | | |
| 0.0025 | Weakly acidic, | 9 | 99.9 | Good |
| | cationic | | | |
| 0.0025 + | Strong acid | 6 | 99.8 | Good |
| 0.1 | cation + FS | | | |
| 0.0025 + | Strong base | 53 | 98.5 | Fair |
| 0.1 | anionic + FS | | | |
| 0.0025 + | Strong acid | 3 | 99.9 | Good |
| 0.1 | cationic + FS | | | |
| 0.0025 + 0.1 | Divinylbenzene | 111 | 96.8 | Poor |
| | hydrophobic | | | |
| | polyaromatic + | | | |
| | FS | | | |
| 0.0025 + | Weakly acidic, | 2.1 | 99.9 | Good |
| 0.1 | cationic + FS | | | |

Abatement Comments to table 6:
100 = Great;
>99.9 = Good;
>98-<99 = Fair; and
<98 = Poor.

Scale is related to the odor threshold of ammonia. Sited 5-50 ppm is OSHA noticeable odor. Target is below 5 ppm ammonia remaining.

In table 6, we analyzed the abatement of 300 ppm ammonia at a timing of 5 min and 15 minutes after spiking. The strong base anionic exchange resin was not able to abate to reduced level of ammonia. The weak acid and strong acid cationic exchange resins were able to abate the ammonia. The strong acid cationic exchange resin was able to abate significantly better than the weak acid cationic exchange resin under the shorter spiking and sampling times.

TABLE 6

| | | | % |
|---|---|---|---|
| Sample | 300 ppm, 15 min | 300 ppm, 5 min | Abatement |
| Control | 286 | 288 | |
| Fresh Step | 0 | 3.6 | 99 |
| Weak acid ion | 0 | 4.8 | 98 |
| exchange resin | | | |
| 0.005 g | | | |
| Weak acid ion | 0 | 2.5 | 99 |
| exchange resin | | | |
| (0.005) + FS | | | |
| Strong base anionic | 216 | 293 | 0 |
| exchange resin | | | |
| (0.005) | | | |
| Strong base anionic | 0 | 3.5 | 99 |
| exchange resin | | | |
| (0.005) + FS | | | |
| Strong acid ion | 0 | 0 | 100 |
| exchange resin | | | |
| (0.005) | | | |
| Strong acid ion | 0 | 0 | 100 |
| exchange resin | | | |
| (0.005) + FS | | | |

TABLE 7

| Potential Samples | Component | Comments |
|---|---|---|
| AMBERJET 1000 H Ion | Strong Acid cation exchanger, sulfonic acid, | |
| Exchange Resin | H+ form Sty/DVB | |
| AMBERJET 1600 H Ion | Strong Acid cation exchange polymer, | |
| Exchange Resin | sulfonic acid, hydrogen ion form | |
| AMBERLITE BDDry Ion | Strong Acid cation exchange polymer, | odorless |
| Exchange Resin | hydrogen ion form | amber |
| AMBERLITE XAD4 | Divinylbenzene Hydrophobic polyaromatic | odorless |
| Polymeric Adsorbent | removal of solvents and low mw species | white |
| AMBERLITE IRA96 Resin | weak base anion resin Modified tert-amine | tan |
| | divinylbenzene styrene copolymer | |
| AMBERLITE IRP64 | Insoluble, weakly acidic, H+ form, cation | |
| | exchange resin US2008/0058428 A1, | |
| | Polacriliex resin | |
| AMBERLITE FPA 53 | Modified IER (weak base) adsorbs small | tan clear |
| | molecule responsible for malodor (mostly | amine odor |
| | acids)-AP/DEO | not used |
| DOWEX 1x2 or 1x8 | Strong Base Type 1 Anion, cationic function | 200-400 |
| | Cl_ STY/DVB | mesh (74-37 |
| | | microns) |
| DOWEX 50Wx2 or 50Wx8 | Strong Acid cation resin containing 2 or 8% | 200-400 |
| | divinylbenzene | mesh (74-37 |
| | | microns) |
| AMBERJET 4200 Cl | | amber |
| | | amine odor |
| | | not used |
| AMBERLITE XAD-16 | Nonionic, hydrophobic, xl, small molecules | |
| | antibiotic recovery | |
| AMBERLYST 121 | Strong Acid cationic resin 80% moisture | |
| | capacity | |

Data indicate that strong to weak acid ion exchange resins have excellent odor abatement compared to strong base ion exchange resins or nonionics.

1. A method for reducing odor in waste products by adding an acidic ion exchange resin to an absorbent which is in contact with said waste products.

2. The method of claim 1 in which the acidic ion exchange resin is added to the absorbent in an amount from 0.1 to 50 wt % of the absorbent plus ion exchange resin.

3. The method of claim 2 in which the acidic ion exchange resin is a crosslinked acrylic resin, styrenic resin or a combination thereof.

4. The method of claim 3 in which the absorbent is cat litter.

5. The method of claim 4 in which the acidic ion exchange resin is added to the absorbent in an amount from 0.5 to 20 wt % of the absorbent plus ion exchange resin.

6. The method of claim 5 in which the acidic ion exchange resin comprises sulfonic or carboxylic acid functionality.

7. The method of claim 6 in which the acidic ion exchange resin is a weak acid ion exchange resin.

8. The method of claim 6 in which the acidic ion exchange resin is a strong acid ion exchange resin.
